(19) United States
(12) Patent Application Publication
(10) Pub. No.: US 2022/0098556 A1
(43) Pub. Date: Mar. 31, 2022

(54) SEED CULTURE PROCESS FOR AAV PRODUCTION

(71) Applicant: Biogen MA Inc., Cambridge, MA (US)

(72) Inventors: Terrence Dobrowsky, Waltham, MA (US); Jenny Wangyuanzhen Shupe, Littleton, MA (US)

(21) Appl. No.: 17/425,618

(22) PCT Filed: Jan. 24, 2020
(86) PCT No.: PCT/US2020/014972

§ 371 Date: Jul. 23, 2021

Related U.S. Application Data
(60) Provisional application No. 62/796,826, filed on Jan. 25, 2019.
(60) Provisional application No. 62/824,364, filed on Mar. 27, 2019.
(60) Provisional application No. 62/847,838, filed on May 14, 2019.

(51) Int. Cl. C12N 7/00; C12M 1/00

(52) U.S. Cl. CPC C12N 7/00; C12N 2710/10051; C12M 29/10

(57) ABSTRACT

Provided herein are methods of producing adeno-associated virus (AAV) comprising culturing AAV producer cell lines in a seed culture followed by an AAV production culture.

### FIELD OF THE INVENTION

The present disclosure relates to methods of culturing a seed cell culture to achieve high viable cell densities in order to improve the production of adeno-associated virus (AAV) particles.

### BACKGROUND OF THE INVENTION

Gene therapy is a promising cure for many diseases such as genetic disorders. Adeno-associated vectors (AAV) have been the gene delivery vehicle of choice in the gene therapy field. To date, the commercial scale of AAV manufacturing using suspension cell culture has been limited at low cell densities. Thus, there is a need for improved methods for culturing AAV-producing suspension cells.

### BRIEF SUMMARY OF THE INVENTION

The present disclosure addresses the need for improved methods for culturing AAV-producing cells, e.g., in suspension, e.g., in a bioreactor.

In some aspects, provided herein is a method of producing AAV comprising:
(a) culturing mammalian cells (e.g., cells described herein, e.g., HeLa cells, CHO cells, HEK-293 cells, VERO cells, NS0 cells, PER.C6 cells, Sp2/0 cells, BHK cells, MDCK cells, MDBK cells, or COS cells) in a N-1 culture vessel, wherein the cells comprise one or more AAV components;
(b) inoculating a N culture vessel with the cells obtained from step (a) at a predetermined dilution factor; and
(c) culturing the cells in the N culture vessel under conditions that allow production of the AAV, thereby producing the AAV.

In some embodiments, step (a) further comprises removing waste products and/or supplementing with fresh nutrients, e.g., using perfusion (e.g., alternating tangential flow (ATF) perfusion) or using a fed batch process.

In some embodiments, the step (a) comprises using ATF perfusion. In other embodiments, the step (a) comprises using a fed batch process.

In some embodiments, the step (a) comprises using a fed batch process and wherein:
(i) the predetermined dilution factor in the step (b) is about ⅙ to about ⅓, e.g., about ⅕, of the cell density of the cells obtained from step (a); and/or
(ii) the N culture vessel seeding density of step (b) is about 1E6 to about 3E6 viable cells/mL, e.g., about 2E6 to about 3E6 viable cells/mL.

In some embodiments, the step (a) comprises using ATF perfusion and wherein:
(i) the predetermined dilution factor in the step (b) is about 1/10 to about ⅕ of the cell density of the cells obtained from step (a); and/or
(ii) the N culture vessel seeding density of step (b) is about 3E6 to about 1E7 viable cells/mL.

In some embodiments, the predetermined dilution factor in the step (b) is about 1/20 to about ½ of the cell density of the cells obtained from step (a). In embodiments, the predetermined dilution factor in the step (b) is about 1/20 to about ½, about 1/20 to about ⅓, about 1/20 to about ¼, about 1/20 to about ⅕, about 1/20 to about 1/10, about 1/10 to about ½, about 1/10 to about ⅓, about 1/10 to about ¼, about 1/10 to about ⅕, about 1/10 to about ⅙, about 1/15 to about ⅕, about ⅙ to about ½, about ⅙ to about ⅓, about ⅙ to about ¼, or about ½, about ⅓, about ¼, about ⅕, about ⅙, about 1/7, about ⅛, about 1/9, about 1/10, about 1/11, about 1/12, about 1/13, about 1/14, or about 1/15. In some embodiments, the predetermined dilution factor in the step (b) is about ⅙ to about ⅓, e.g., ⅕. In some embodiments, the predetermined dilution factor in the step (b) is about 1/10 to about ⅕.

In some embodiments, the predetermined dilution factor in the step (b) is about ⅙ to about ⅓, e.g., about ⅕ of the cell density of the cells obtained from step (a),

optionally wherein the step (a) comprises using a fed batch process, e.g., a fed batch process described herein; and/or

optionally wherein the step (a) does not comprise using perfusion, e.g., ATF perfusion.

In some embodiments, the predetermined dilution factor in the step (b) is about 1/10 to about ⅕ of the cell density of the cells obtained from step (a),

optionally wherein the step (a) comprises using perfusion, e.g., ATF perfusion, e.g., ATF perfusion described herein.

In some embodiments, the step (c) is performed for less than 6 days, e.g., less than 5, 4, 3, 2, 1 day or less. In embodiments, the step (c) is performed for 5, 4, 3, 2, 1 day or less, e.g., 2-5 days, e.g., 2, 3, 4, or 5 days.

In some embodiments, the step (a) comprises culturing the cells to achieve an N-1 cell density of at least 1E7 viable cells/mL, e.g., about 1E7 to about 4E8 viable cells/mL, about 1E7 to about 3E8 viable cells/mL, about 1E7 to about 2E8 viable cells/mL, e.g., about 2E7 to about 1E8. In embodiments, the step (a) comprises culturing the cells to achieve an N-1 cell density of about 1E7 to about 2E7, about 2E7 to about 3E7, about 3E7 to about 4E7, about 4E7 to about 5E7, about 5E7 to about 6E7, about 6E7 to about 7E7, about 7E7 to about 8E7, about 8E7 to about 9E7, about 9E7 to about 1E8, or about 1E8 to about 2E8 viable cells/mL.

In some embodiments, the N culture vessel seeding density of step (b) is about 5E5 to about 2E7 viable cells/mL, e.g., about 5E5 to about 7.5E5, about 7.5E5 to about 1E6, about 1E6 to about 2E6, about 1E6 to about 3E6, about 1E6 to about 4E6, about 1E6 to about 5E6, about 2E6 to about 4E6, about 2E6 to about 3E6, about 3E6 to about 1E7, about 3E6 to about 8E6, about 3E6 to about 6E6, about 4E6 to about 1E7, about 5E6 to about 1E7, about 6E6 to about 1E7, about 7E6 to about 1E7, about 8E6 to about 1E7, about 4E6 to about 8E6, about 8E6 to about 1E7, or about 1E7 to about 2E7 viable cells/mL. In some embodiments, the N culture vessel seeding density of step (b) is about 1E6 to about 3E6, e.g., about 2E6 to about 3E6 viable cells/mL. In some embodiments, the N culture vessel seeding density of step (b) is about 3E6 to about 1E7 viable cells/mL.

In some embodiments, the N culture vessel seeding density of step (b) is about 1E6 to about 3E6 viable cells/mL,

optionally wherein the step (a) comprises using a fed batch process (e.g., a fed batch process described herein), and

optionally wherein the step (a) does not comprise using perfusion, e.g., ATF perfusion (e.g., an ATF perfusion process described herein).

In some embodiments, the N culture vessel seeding density of step (b) is about 3E6 to about 1E7 viable cells/mL, optionally wherein the step (b) comprises using perfusion, e.g., ATF perfusion (e.g., an ATF perfusion process described herein).

In some embodiments, the N-1 culture is about 2 L to about 25,000 L (e.g., about 2 L to about 10 L, or about 50 L to about 100 L, or about 2000 L to about 25,000 L) in volume.

In some embodiments, the N culture is about 2000 L to about 50,000 L in volume (e.g., about 2000 L to about 10,000 L, about 10,000 L to about 25,000 L, about 25,000 L to about 50,000 L, about 10,000 L to about 50,000 L, about 10,000 L to about 40,000 L, or about 10,000 L to about 30,000 L).

In some embodiments, the cells comprise HeLa cells, CHO cells, HEK-293 cells, VERO cells, NS0 cells, PER.C6 cells, Sp2/0 cells, BHK cells, MDCK cells, MDBK cells, or COS cells, e.g., HeLa PCLs, CHO PCLs, HEK-293 PCLs, VERO PCLs, NS0 PCLs, PER.C6 PCLs, Sp2/0 PCLs, BHK PCLs, MDCK PCLs, MDBK PCLs, or COS PCLs. In some embodiments, the cells comprise human cells, e.g., human PCLs. In some embodiments, the cells comprise HeLa cells, e.g., HeLa PCLs. In some embodiments, the cells comprise CHO cells, e.g., CHO PCLs. In some embodiments, the cells comprise HEK-293 cells, e.g., HEK-293 PCLs.

In some embodiments, the step (a) and step (c) comprise culturing the cells in suspension.

In some embodiments, the ATF perfusion is performed at a flow rate of about 3 mL/min/fiber to about 15 mL/min/fiber, e.g., about 3 mL/min/fiber to about 12 mL/min/fiber, about 5 mL/min/fiber to about 10 mL/min/fiber.

In some embodiments, the cell specific perfusion rate (CSPR) in the step (a) is at least about 0.02 nL/cell/day, e.g., about 0.02 nL/cell/day to about 0.1 nL/cell/day, e.g., about 0.03 nL/cell/day to about 0.06 nL/cell/day, e.g., about 0.03 nL/cell/day, about 0.04 nL/cell/day, about 0.05 nL/cell/day, or about 0.06 nL/cell/day.

In some embodiments, the step (a) is performed for 5 to 12 days, e.g., about 5, 6, 7, 8, 9, 10, 11, or 12 days.

In some embodiments, the method further comprises a step (d) collecting the AAV produced by the cells in the N culture vessel.

In accordance with any methods described herein, in some embodiments, the AAV component comprises one or more (any combination) of the following:
(a) a nucleic acid sequence comprising a transgene;
(b) a nucleic acid sequence comprising an inverted terminal repeat (ITR), e.g., one or two ITRs;
(c) a nucleic acid sequence encoding one or more AAV replication and/or packaging proteins (e.g., encoded by the AAV rep gene);
(d) a nucleic acid sequence encoding one or more AAV structural capsid proteins (e.g., encoded by the AAV cap gene, e.g., VP1, VP2, or VP3 protein);
(e) one or more AAV replication and/or packaging proteins;
(f) one or more AAV structural capsid proteins; and/or
(g) one or more Ad5 helper function components (e.g., Ad5 helper virus components described herein, e.g., E1a, E1b, E2a, E4Orf6, or VA RNA; and/or Ad5 helper virus), optionally where any combination of (a)-(d) or (g) are disposed on the same nucleic acid molecule or on separate nucleic acid molecules (e.g., disposed on one, two, three, or four separate nucleic acid molecules).

In some embodiments, the step of culturing the cells in the N culture vessel under conditions that allow production of the AAV, thereby producing the AAV, step (c), further comprises providing one or more AAV components, e.g., AAV components described herein, e.g., AAV components (a)-(g) described herein, to the N culture. In some embodiments, the step (c) further comprises providing a helper virus, e.g., an adenovirus (e.g., Ad5 helper virus) or a herpes virus, to the N culture vessel. In some embodiments, the step (c) further comprises providing a Ad5 helper virus comprising a transgene and/or ITRs, to the N culture. In some embodiments, the step (c) further comprises providing one or more additional AAV components (e.g., AAV components described herein, e.g., AAV components (a)-(g) described herein), e.g., AAV components that are not already in the N-1 culture, to the N culture vessel.

In some embodiments, the method comprises producing about 1E4 to about 1E5 AAV vector genomes/cell (vg/cell). In some embodiments, the method comprises producing about 1E9 to about 1E11 AAV vector genomes/mL (vg/mL), e.g., about 2E9 to about 6E10 vg/mL, or about 5E9 to about 6E10 vg/mL, or about 1E10 to about 6E10 vg/mL.

In some embodiments, the transgene encodes a therapeutic polypeptide.

In some embodiments, the fed batch process comprises supplementing the N-1 culture periodically with a supplement (e.g., fresh media, amino acids, and/or glucose). In some embodiments, the fed batch process comprises supplementing the N-1 culture once a day, every 2 days, or twice a day. In some embodiments, the fed batch process comprises supplementing the N-1 culture once a day. In some embodiments, the amount (e.g., volume, concentration, and/or feed %) of the supplement is determined based on the integrated cell growth (ICG) of the N-1 culture. In some embodiments, the amount (e.g., volume, concentration, and/or feed %) of the supplement is determined using a feed addition slope of about 0.0002 mL*day/cells to about 0.02 mL*day/cells, e.g., about 0.0005 mL*day/cells to about 0.005 mL*day/cells.

### BRIEF DESCRIPTION OF THE DRAWINGS

FIG. 1 is a panel of graphs that shows the viable cell density (VCD) (e6 cell/mL) (bottom three panels) and the viability (%) (top three panels) at different days of culture in an N-1 seed culture using a fed batch process. The different lines in each panel correspond to different feed addition slopes. The feed addition slopes (also referred to herein as slopes) are shown as normalized values relative to a control slope.

FIG. 2 is a schematic showing an exemplary ATF perfusion setup.

FIGS. 3A-3N are graphs showing various parameters achieved in an N-1 seed culture using ATF perfusion. FIG. 3A shows viable cell density (e6 cells/mL) vs. culture day. FIG. 3B shows normalized concentrations of amino acids using different media at different culture days. FIG. 3C shows viable cell density at different flow rates versus culture day. FIG. 3D shows viable cell density versus culture day. FIG. 3E shows viability (%) vs. culture day. FIG. 3F shows growth rate vs. culture day. FIG. 3G shows pH vs. culture day. FIG. 3H shows pCO₂ (mmHg) vs. culture day. FIG. 3I shows relative glutamine concentration vs. culture day. FIG. 3J shows relative glucose concentration vs. culture day. FIG. 3K shows relative glutamate concentration vs. culture day. FIG. 3L shows relative lactate concentration vs. culture day. FIG. 3M shows relative ammonia concentration vs. culture day. FIG. 3N shows relative lactate dehydrogenase (LDH) concentration vs. culture day. For FIGS. 3C-3N, the three different lines represent three different cell clones.

FIGS. 4A-4C are graphs showing the viable cell density (e6 cells/mL) (FIG. 4A), overall growth rate (FIG. 4B), and % viability (FIG. 4C) vs. culture day when the N-1 culture was performed at lab scale and pilot scale. The different lines indicate experiments performed at lab scale and at pilot scale.

FIG. 5 is a graph showing the effect of seed density on the normalized AAV genome titer in the N production culture, with a higher seed density (N-1 culture into N culture) leading to a higher AAV genome titer in the N culture. The top bar shows the normalized AAV genome titer when using a lower seed density (about 5E5 cells/mL), and the bottom bar shows the normalized AAV genome titer when using a higher seed density (about 2E6 cells/mL). The AAV genome titer achieved using higher seed density was about 11 times the titer achieved with the lower seed density condition. The normalization is relative to the AAV genome titer achieved with the lower seed density condition.

### DETAILED DESCRIPTION

To date, the commercial scale of AAV manufacturing using suspension cell culture has been limited at low cell densities, primarily due to two reasons: the necessity of significantly diluting the seed culture with fresh production medium to maintain high specific productivity, and the generally low maximal final cell density achieved through traditional batch culture in an N-1 seed culture step. The methods provided herein address these challenges by achieving a higher cell density while still maintaining a high viability and consistent growth rate.

Provided herein are methods of producing AAV comprising culturing a seed culture (N-1 culture) and subsequently seeding cells from that seed culture into an AAV production culture (N culture). The invention is based at least in part on the surprising discovery of methods that significantly increase the viable cell density in the N-1 culture and that seeding the N culture at a higher density can increase AAV titers in the N culture. For example, the methods provided herein contemplate use of alternating tangential flow (ATF) perfusion or fed batch methods during the N-1 culture to significantly increase the viable cell density in the N-1 culture. In some examples, using the methods described herein, a viable cell density in the N-1 step of at least about 1E7 viable cells/mL (e.g., at least about 1E8 viable cells/mL) can be achieved. The methods described herein mitigate a challenge in the bioreactor production field of having to significantly dilute the seed culture with fresh production medium in order to maintain high specific productivity. The methods described herein also mitigate the challenge surrounding the generally low maximal final cell density achieved through traditional batch (as opposed to perfusion, e.g., ATF perfusion, or fed batch) culture. Advantageously, the methods described herein achieve a high viable cell density while also maintaining high viability and consistent growth rate. Such improvements in the N-1 culture methodology enable higher (e.g., 2-10-fold higher) cell densities during AAV production (N culture).

### Definitions

It is to be noted, unless otherwise clear from the context, that the term “a” or “an” entity refers to one or more of that entity; for example, “an amino acid,” is understood to represent one or more amino acids. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone). The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

It is understood that wherever embodiments are described with the language “comprising,” otherwise analogous embodiments described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments of the disclosure, which can be had by reference to the specification as a whole. Accordingly, the terms defined immediately below are more fully defined by reference to the specification in its entirety.

The terms “media”, “medium”, “cell culture medium”, “culture medium”, “tissue culture medium”, “tissue culture media”, and “growth medium” as used herein refer to a solution containing nutrients which nourish growing cultured eukaryotic cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium”—a serum-free medium that contains no proteins, hydrolysates or components of unknown composition. Defined media are free of animal-derived components and all components have a known chemical structure. One of skill in the art understands a defined medium can comprise recombinant glycoproteins or proteins, for example, but not limited to, hormones, cytokines, interleukins and other signaling molecules.

The term “basal media formulation” or “basal media” as used herein refers to any cell culture media used to culture cells that has not been modified either by supplementation, or by selective removal of a certain component.

The terms “culture”, “cell culture” and “eukaryotic cell culture” as used herein refer to a eukaryotic cell population, either surface-attached or in suspension that is maintained or grown in a medium (see definition of “medium” below) under conditions suitable to survival and/or growth of the cell population. As will be clear to those of ordinary skill in the art, these terms as used herein can refer to the combination comprising the mammalian cell population and the medium in which the population is suspended.

The term “batch culture” as used herein refers to a method of culturing cells in which all the components that will ultimately be used in culturing the cells, including the medium (see definition of “medium” below) as well as the cells themselves, are provided at the beginning of the culturing process. A batch culture is typically stopped at some point and the cells and/or components in the medium are harvested and optionally purified.

The term “fed-batch culture” as used herein refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. The provided components typically comprise nutritional supplements for the cells which have been depleted during the culturing process. A fed-batch culture is typically stopped at some point and the cells and/or components in the medium are harvested and optionally purified.

The term “perfusion culture” as used herein refers to a method of culturing cells in which additional components are provided continuously or semi-continuously to the culture subsequent to the beginning of the culture process. The provided components typically comprise nutritional supplements for the cells which have been depleted during the culturing process. A portion of the cells and/or components in the medium are typically harvested on a continuous or semi-continuous basis and are optionally purified.

“Growth phase” of the cell culture refers to the period of exponential cell growth (the log phase) where cells are generally rapidly dividing. During this phase, cells are cultured for a period of time, usually between 1-4 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. In some embodiments, during the growth phase, cells are cultured in nutrient medium containing the necessary additives generally at about 25°-40° C., in a humidified, controlled atmosphere, such that optimal growth is achieved for the particular cell line. In embodiments, cells are maintained in the growth phase for a period of about between one and seven days, e.g., between two to six days, e.g., six days. The length of the growth phase for the particular cells can be determined without undue experimentation. For example, the length of the growth phase will be the period of time sufficient to allow the particular cells to reproduce to a viable cell density within a range of about 20% -80% of the maximal possible viable cell density if the culture was maintained under the growth conditions. In some embodiments, “maximum growth rate” refers to the growth rate of the specific cell line/clone measured during its exponential growth phase, while the cells are in fresh culture medium (e.g., measured at a time during culture when nutrients are sufficient and there is not any significant inhibition of growth from any components of the culture).

The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time.

The term “cell density” as used herein refers to that number of cells present in a given volume of medium.

The term “bioreactor” or “culture vessel” as used herein refers to any vessel used for the growth of a mammalian cell culture. The bioreactor can be of any size so long as it is useful for the culturing of mammalian cells.

As used herein, the term “bioreactor run” can include one or more of the lag phase, log phase, or plateau phase growth periods during a cell culture cycle.

The term “first culture vessel,” “N-1 culture vessel,” “N-1 seed-train culture vessel,” “N-1 vessel,” “first bioreactor,” “N-1 bioreactor,” “N-1 seed-train bioreactor” as used herein refers to a culture vessel that is immediately before the N culture vessel (production culture vessel) and is used to grow the cell culture to a high viable cell density for subsequent inoculation into N (production) culture vessel. The cell culture to be grown in the N-1 culture vessel may be obtained after culturing the cells in several vessels prior to the N-1 culture vessel, such as N-4, N-3, and N-2 vessels.

The terms “N culture vessel,” “second culture vessel,” “production culture vessel,” “N vessel,” “N bioreactor,” “second bioreactor,” “production bioreactor” as used herein refers to the bioreactor after the N-1 bioreactor and is used in the production of the AAV.

The term “seeding” as used herein refers to the process of providing a cell culture to a bioreactor or another vessel. In one embodiment, the cells have been propagated previously in another bioreactor or vessel. In another embodiment, the cells have been frozen and thawed immediately prior to providing them to the bioreactor or vessel. The term refers to any number of cells, including a single cell.

The term “AAV titer” as used herein refers to the number of viral genomes per ml (vg/ml) or the vector genomes per cell (vg/cell). In embodiments, the vg/mL, can be determined by standard methods, including but not limited to direct QPCR of purified vector particles, FACS, silver stain, etc. In embodiments, the vg/cell can be determined by standard methods, e.g., including but not limited to dot blot, quantitative PCR or ddPCR, spectroscopy, or fluorimetry.

The term “Ad5 helper function components” as used herein encompasses the Ad5 helper virus (e.g., wildtype or recombinantly engineered Ad5 helper virus) as well as various Ad5 helper virus genes/factors, including but not limited to E1a, E1b, E2a, E4Orf6, and VA RNA.

The term “amino acid” as used herein refers any of the twenty standard amino acids, i.e., glycine, alanine, valine, leucine, isoleucine, methionine, proline, phenylalanine, tryptophan, serine, threonine, asparagine, glutamine, tyrosine, cysteine, lysine, arginine, histidine, aspartic acid and glutamic acid, single stereoisomers thereof, and racemic mixtures thereof. The term “amino acid” can also refer to the known non-standard amino acids, e.g., 4-hydroxyproline, ε-N,N,N-trimethyllysine, 3-methylhistidine, 5-hydroxylysine, O-phosphoserine, γ-carboxyglutamate, ε-N-acetyllysine, ω-N-methylarginine, N-acetylserine, N,N,N-trimethylalanine, N-formylmethionine, γ-aminobutyric acid, histamine, dopamine, thyroxine, citrulline, ornithine, β-cyanoalanine, homocysteine, azaserine, and S-adenosylmethionine. In some embodiments, the amino acid is glutamate, glutamine, lysine, tyrosine or valine. In some embodiments, the amino acid is glutamate or glutamine.

The term “nutrient media,” “feed media,” “feed,” “total feed,” and “total nutrient media” as used herein can be used interchangeably, and include a “complete” media used to grow, propagate, and add biomass to a cell line. Nutrient media is distinguished from a substance or simple media which by itself is not sufficient to grow and propagate a cell line. Thus, for example, glucose or simple sugars by themselves are not nutrient media, since in the absence of other required nutrients, they would not be sufficient to grow and propagate a cell line. One of skill in the art can appreciate that cells may continue to grow, live and propagate in the presence of incomplete media, but become instable and/or greatly reduce their growth rate. Thus, in some embodiments, the term “nutrient media” includes a media sufficient to grow, propagate, and add biomass to a cell line without a loss in stability, growth rate, or a reduction of any other indicators of cellular health for a period of at least 2 days, 3 days, 4 days, 5 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, or 18 weeks. In some embodiments, the term “nutrient media” includes a media which may lack one or more essential nutrients, but which can continue to grow, propagate, and add biomass to a cell line without a loss in stability, growth rate, or a reduction of any other indicators of cellular health for a period of at least 2 days, 3 days, 4 days, 5 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, or 18 weeks.

In some embodiments, the nutrient media is a cell culture media. Optimal cell culture media compositions vary according to the type of cell culture being propagated. In some embodiments, the nutrient media is a commercially available media. In some embodiments, the nutrient media contains e.g., inorganic salts, carbohydrates (e.g., sugars such as glucose, galactose, maltose or fructose), amino acids, vitamins (e.g., B group vitamins (e.g., B12), vitamin A vitamin E, riboflavin, thiamine and biotin), fatty acids and lipids (e.g., cholesterol and steroids), proteins and peptides (e.g., albumin, transferrin, fibronectin and fetuin), serum (e.g., compositions comprising albumins, growth factors and growth inhibitors, such as, fetal bovine serum, newborn calf serum and horse serum), trace elements (e.g., zinc, copper, selenium and tricarboxylic acid intermediates), hydrolysates (hydrolyzed proteins derived from plant or animal sources), and combinations thereof. Examples of nutrient medias include, but are not limited to, basal media (e.g., MEM, DMEM, GMEM), complex media (RPMI 1640, Iscoves DMEM, Leibovitz L-15, Leibovitz L-15, TC 100), serum free media (e.g., CHO, Ham F10 and derivatives, Ham F12, DMEM/F12). Common buffers found in nutrient media include PBS, Hanks BSS, Earles salts, DPBS, HBSS, and EBSS. Media for culturing mammalian cells are well known in the art and are available from, e.g., Sigma-Aldrich Corporation (St. Louis, Mo.), HyClone (Logan, Utah), Invitrogen Corporation (Carlsbad, Calif.), Cambrex Corporation (E. Rutherford, N.J.), Irvine Scientific (Santa Ana, Calif.), and others. Other components found in nutrient media can include ascorbate, citrate, cysteine/cystine, glutamine, folic acid, glutathione, linoleic acid, linolenic acid, lipoic acid, oleic acid, palmitic acid, pyridoxal/pyridoxine, riboflavin, selenium, thiamine, and transferrin. One of skill in the art will recognize that there are modifications to nutrient media which would fall within the scope of this invention.

The terms “polypeptide” or “protein” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. If a single polypeptide is the discrete functioning unit and does require permanent physical association with other polypeptides in order to form the discrete functioning unit, the terms “polypeptide” and “protein” as used herein are used interchangeably. If discrete functional unit is comprised of more than one polypeptide that physically associate with one another, the term “protein” as used herein refers to the multiple polypeptides that are physically coupled and function together as the discrete unit. The term “protein” as used herein is intended to encompass a singular “protein” as well as plural “proteins.” Thus, as used herein, terms including, but not limited to “peptide,” “polypeptide,” “amino acid chain,” or any other term used to refer to a chain or chains of amino acids, are included in the definition of a “protein,” and the term “protein” may be used instead of, or interchangeably with, any of these terms. The term further includes proteins which have undergone post-translational modifications, for example, glycosylation, acetylation, phosphorylation, amidation, derivatization by known protecting/blocking groups, proteolytic cleavage, or modification by non-naturally occurring amino acids. Proteins also include polypeptides which form multimers, e.g., dimers, trimers, etc. The term protein also includes fusions proteins, e.g., a protein that is produced via a gene fusion process in which a protein (or fragment of a protein) is attached to an antibody (or fragment of antibody).

### AAV

The methods provided herein relate to the production of adeno-associated viruses (AAV), which can be used as gene delivery vehicles. In some aspects, wild-type AAV is engineered to include a transgene, e.g., a transgene encoding a therapeutic polypeptide, e.g., therapeutic protein. In some embodiments, such engineered AAV can be administered to a subject in need of the therapeutic transgene/polypeptide, and upon administration, the therapeutic polypeptide can be expressed in the subject's own cells. The methods described herein are applicable to an AAV comprising any transgene(s).

### AAV Producer Cell Lines

Various components/machinery are needed for AAV production, including for example, components for replication, packaging, and structural components of the capsid. For example, the AAV Rep gene encodes four proteins that are involved in packaging and replication, and the cap gene encodes three structural capsid proteins (called VP1, VP2, and VP3). Wild-type AAV is replication deficient and requires co-infection of cells by a helper virus, e.g., a herpes virus or adenovirus, e.g., Ad5 virus, in order to replicate. For example, Ad5 virus supplies Ad5 helper function factors/genes, such as E1a, E1b, E4Orf6, E2a and/or virus-associated (VA) RNA, that mediate AAV replication. See, e.g., Nayak et al. J Virol. 81.5(2007):2205-12. Recombinant AAV vectors permit integration of a gene of interest, or transgene, into a viral vector such that the transgene is transmitted, encoded, and/or expressed by the viral machinery. In some cases, a recombinant AAV vector comprises inverted terminal repeats (ITRs) that serve as origins of replication and/or packaging. In some embodiments, the recombinant AAV vector comprises a transgene flanked by ITRs (one ITR on either side of the transgene). See, e.g., Carter B. Adeno-Associated Virus and AAV Vectors for Gene Delivery, in Gene and Cell Therapy, 4th Edition, N.S. Templeton, Editor. 2015, CRC Press.

AAV producer cell lines (PCLs) can be generated that contain one or more components required for AAV production. An exemplary list of AAV components includes the following:
(a) a nucleic acid sequence comprising a transgene;
(b) a nucleic acid sequence comprising an inverted terminal repeat (ITR), e.g., one or two ITRs, e.g., where the two ITRs flank one or more additional AAV components (e.g., a transgene);
(c) a nucleic acid sequence encoding one or more AAV replication and/or packaging proteins (e.g., encoded by the AAV rep gene);
(d) a nucleic acid sequence encoding one or more AAV structural capsid proteins (e.g., encoded by the AAV cap gene, e.g., VP1, VP2, or VP3 protein);
(e) one or more AAV replication and/or packaging proteins;
(f) one or more AAV structural capsid proteins; and/or
(g) one or more helper virus components, e.g., Ad5 helper function components (e.g., E1a, E1b, E4Orf6, E2a and/or VA RNA; or Ad5 helper virus),
   for example in any combination. Thus, as used herein, an “AAV PCL” or “AAV producer cell line” refers to a cell, e.g., a cell described herein, that comprises one or more of (a)-(g) above. An “AAV PCL” is used interchangeably herein with a “cell that comprises one or more AAV components.”

In some embodiments, any combination of the AAV components (e.g., (a)-(d) or (g) above) are disposed on the same nucleic acid molecule or on separate nucleic acid molecules (e.g., disposed on one, two, three, or four or more separate nucleic acid molecules). In some embodiments, the transgene is flanked on both sides by an ITR.

In some embodiments, a cell described herein, e.g., a PCL described herein, comprises any combination of (a)-(g). In some embodiments, the PCL can comprise recombinant AAV vector(s) and/or Rep and/or cap genes, e.g., stably integrated into a permissive host cell. In some embodiments, the PCL comprises a nucleic acid sequence encoding a transgene and a nucleic acid sequence comprising one or more ITRs, e.g., where the transgene is flanked on either side by one ITR. In other embodiments, a PCL comprises a packaging cell line that comprises rep and/or cap genes but not the vector/transgene (and the vector/transgene can be supplied by a separate virus, e.g., recombinant adenovirus, e.g., Ad5). PCLs comprising other combinations of AAV components can be made. Any such PCLs and other variations of PCLs can be used in connection with the methods described herein. In some examples, AAV production can be induced by infection with a helper virus, such as Ad5, e.g., in the N culture.

### Culture of AAV Producer Cell Lines (PCLs)

Provided herein are methods of culturing cells, e.g., AAV producer cell lines (PCLs), in a N-1 culture vessel to achieve a high viable cell density, using the N-1 culture cells to seed a N culture vessel at a particular seeding density, and culturing the seeded cells in the N culture vessel under conditions that permit production of AAV.

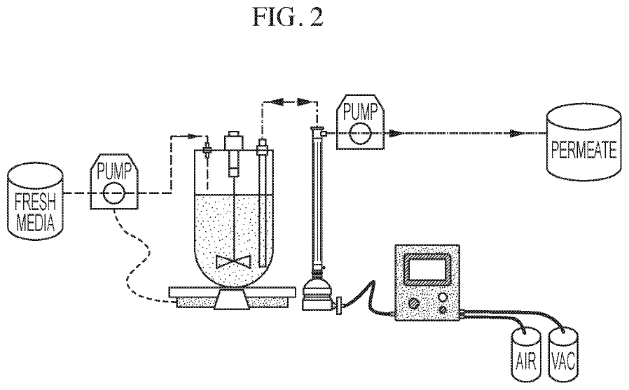

In some embodiments, the step of culturing the AAV PCLs in the N-1 culture vessel comprises removing waste and/or supplementing with nutrients, e.g., by using perfusion, e.g., alternating tangential flow (ATF) perfusion. ATF is made up of a filter system that provides a way to continuously or semi-continuously exchange the cell culture media, e.g., by continuously/semi-continuously supplementing the culture with fresh media while removing waste, e.g., through a multitude of hollow fibers. An example of the workflow is shown in FIG. 2. The ATF flow rate indicates how fast the cell culture circulates through each hollow fiber. In some embodiments, the diameter of each fiber is about 0.5 mm to about 1.5 mm, e.g., about 0.5 mm, about 1 mm, or about 1.5 mm. Without wishing to be bound by theory, it is believed that a higher flow rate tends to increase the amount of shear stress on the cells. And, too low of a flow rate may increase (1) the risk of the cell culture being exposed to low oxygen environments for a prolonged period of time and (2) the possibility of filter fouling due to insufficient back flushing. The methods described herein comprise performing the ATF perfusion at a flow rate that minimizes shear stress on the cells, yet provides sufficient oxygen to the cells. In some embodiments, the ATF perfusion is performed at a flow rate of about 3 mL/min/fiber to about 15 mL/min/fiber, e.g., about 5 mL/min/fiber to about 10 mL/min/fiber, about 5 mL/min/fiber to about 8 mL/min/fiber, about 7 mL/min/fiber to about 10 mL/min/fiber, or about 8 mL/min/fiber to about 10 mL/min/fiber. In some embodiments, the ATF perfusion is performed at a flow rate of about 3 mL/min/fiber, about 4 mL/min/fiber, about 5 mL/min/fiber, about 6 mL/min/fiber, about 7 mL/min/fiber, about 8 mL/min/fiber, about 9 mL/min/fiber, about 10 mL/min/fiber, about 11 mL/min/fiber, about 12 mL/min/fiber, about 13 mL/min/fiber, about 14 mL/min/fiber, or about 15 mL/min/fiber. In other embodiments, the ATF perfusion is performed at a flow rate per fiber of about 50 meters/s to about 320 meters/s, e.g., about 50 meters/s to about 250 meters/s, about 50 meters/s to about 200 meters/s, about 100 meters/s to about 320 meters/s, about 100 meters/s to about 250 meters/s, about 200 meters/s to about 320 meters/s, or about 200 meters/s to about 275 meters/s.

Without wishing to be bound by theory, it is believed that higher cell densities require higher perfusion rates in order to maintain a certain cell growth rate or viability. Since cell density can change over time in a cell culture (e.g., in the N-1 culture described herein), a cell specific perfusion rate is a parameter that quantifies the ATF perfusion rate based on the cell density of the culture. In some embodiments, the methods described herein comprise performing the ATF perfusion in the N-1 culture at a cell specific perfusion rate (CSPR) of about 0.02 nL/cell/day to about 0.1 nL/cell/day, e.g., about 0.02 nL/cell/day to about 0.08 nL/cell/day, about 0.02 nL/cell/day to about 0.06 nL/cell/day, about 0.03 nL/cell/day to about 0.08 nL/cell/day, about 0.03 nL/cell/day to about 0.06 nL/cell/day, e.g., about 0.03 nL/cell/day, about 0.04 nL/cell/day, about 0.05 nL/cell/day, or about 0.06 nL/cell/day.

In other embodiments, the step of culturing the AAV PCLs in the N-1 culture vessel comprises supplementing with nutrients, e.g., using a fed batch culture process comprising supplementing the N-1 culture periodically with a supplement (e.g., fresh media, amino acids, and/or glucose). In some embodiments, the supplementation occurs at least once every two days, e.g., once a day, twice a day, three times a day, four times a day, or more. In some embodiments, the supplementation occurs about once a day. In some embodiments, the supplement comprises an amino acid (e.g., an amino acid described herein), e.g., a mixture of one or more amino acids (e.g., amino acids described herein). In some embodiments, the supplement comprises glutamine. In some embodiments, the supplement comprises glucose. In some embodiments, the supplement comprises glucose and glutamine. In some embodiments, the concentration of supplement added each time is determined based on the viable cell density, e.g., viable cell density determined at the time of supplement addition or just prior to supplement addition. In some embodiments, the amount (e.g., volume and/or concentration) of the supplement is determined based on the integrated cell growth (ICG), e.g., in the N-1 culture. ICG can be calculated based on the area under the cell growth curve and has units of cells*mL⁻¹*day⁻¹. In some embodiments, the amount of supplement added each time (e.g., each day) can be described in terms of feed volume or feed %, where feed % refers to the volume of feed added relative to the volume of the culture, e.g., at time of addition. In some embodiments, the relationship between feed % and ICG can be described in terms of slope (also referred to as feed addition slope), which has units of (mL*day/cells). The slope is equal to feed % divided by ICG. In some embodiments, the slope as used in the fed batch methods described herein is about 0.0002 mL*day/cells to about 0.02 mL*day/cells, or about 0.0005 mL*day/cells to about 0.01 mL*day/cells, or about 0.0005 mL*day/cells to about 0.005 mL*day/cells, or about 0.001 mL*day/cells to about 0.005 mL*day/cells. In some embodiments, the slope as used in the fed batch methods described herein about 1-2-fold (e.g., about 1-fold, about 1.25-fold, about 1.5-fold, about 1.75-fold, or about 2-fold) that of a control slope.

In some embodiments, the supplement comprises a total amino acid concentration of about 50 mM to about 2 M. In embodiments, the supplement comprises a total amino acid concentration of at least 50 mM. In some embodiments, the supplement comprises a total amino acid concentration of about 75 mM to about 500 mM. In some embodiments, the supplement comprises a total amino acid concentration of about 50 mM to about 1 M, about 50 mM to about 500 mM, about 50 mM to about 100 mM, about 50 mM, 55 mM, 60 mM, 65 mM, 70 mM, 80 mM, 90 mM, 100 mM, 110 mM, 120 mM, 130 mM, 140 mM, 150 mM, 160 mM, 170 mM, 180 mM, 190 mM, 200 mM, 250 mM, 300 mM, 350 mM, 400 mM, 500 mM, 750 mM, 800 mM, 1 M, 1.5 M, or 2 M.

In some embodiments, the methods described herein comprise culturing the cells in the

N-1 culture to a viable cell density of at least 1E7 viable cells/mL, e.g., at least about 1E8 viable cells/mL, or at least about 2E8 viable cells/mL, or at least about 3E8 viable cells/mL, or greater. In some embodiments, the methods described herein comprise culturing the cells in the N-1 culture to a viable cell density of about 1E7 to about 3E8 viable cells/mL, e.g., about 1E7 to about 2E8 viable cells/mL, about 1E7 to about 1E8 viable cells/mL, about 2E7 to about 3E8 viable cells/mL, about 2E7 to about 2E8 viable cells/mL, about 2E7 to about 1E8 viable cells/mL, about 5E7 to about 3E8 viable cells/mL, about 5E7 to about 2E8 viable cells/mL, about 5E7 to about 1E8 viable cells/mL, about 1E8 to about 3E8 viable cells/mL, about 1E8 to about 2.5E8 viable cells/mL, about 1E8 to about 2E8 viable cells/mL, about 1.5E8 to about 3E8 viable cells/mL, about 1.5E8 to about 2.5E8 viable cells/mL, about 1.5E8 to about 2E8 viable cells/mL, or about 2E8 to about 3E8 viable cells/mL.

In embodiments, the N-1 culture is maintained at a sufficiently low pCO₂ for a given cell line or clone. In embodiments, the methods described herein provide for CO₂ removal, if/as appropriate for the cell line or clone being cultured. In some embodiments, the methods described herein comprise maintaining the N-1 culture at a pCO2 that is below 120 mmHg, e.g., 100 mmHg or lower, e.g., 90 mmHg, 80 mmHg, 75 mmHg, 60 mmHg, 50 mmHg, 40 mmHg, 30 mmHg, or lower.

In embodiments, the cell growth rate of the N-1 culture is maintained at a growth rate that is close to the maximum growth rate for the cell line/clone being cultured. In embodiments, the methods described herein achieves a growth rate within 15% (e.g., within 15%, 12.5%, 10%, 7.5%, 5%, 2.5%, 1%, or less) of the maximum growth rate of the cell line/clone being cultured. In some embodiments, the maximum growth rate is the growth rate of the particular cell line/clone measured while in fresh culture medium and during its exponential growth phase (e.g., measured at a point in time when nutrients are sufficient and no components in the culture are causing significant growth inhibition). In some embodiments, the overall growth rate depends on the particular cell type/clone being cultured. In some embodiments, the overall growth rate of the cells in the N-1 culture is about 0.2/day to about 1/day, e.g., e.g., about 0.3/day to about 0.8/day, about 0.4/day to about 0.7/day, about 0.5/day to about 0.7/day, about 0.4/day to about 0.6/day, about 0.2/day to about 0.8/day, or about 0.5/day to about 1/day. In some embodiments, the overall growth rate is determined based on the cell density at day zero of the culture.

In some embodiments, the methods described herein achieve a cell viability of at least 80% (e.g., at least 80%, 85%, 90%, 95%, 96%, 97%, 98%, 99%, 99.5%, or greater), e.g., in the N-1 culture, e.g., as measured on any day of the N-1 culture, e.g., using standard methods.

In some embodiments, in accordance with the methods described herein, the pH of the N-1 culture is maintained, e.g., between about 6.5 to about 7.8 (e.g., about 6.5 to about 7.4, about 6.5 to about 7.2, about 6.8 to about 7.4, about 7.0 to about 7.8, about 7.0 to about 7.6, or about 7.2 to about 7.8).

In some embodiments, in accordance with the methods described herein, nutrient concentrations are maintained at a desired level and/or waste products are maintained at a low level, e.g., during the N-1 culture, e.g., during any day of the N-1 culture. In some embodiments, the glucose, glutamine, glutamate, total amino acid, lactate, and/or ammonia concentration in the N-1 culture is maintained at a level within 10-fold (e.g., within 10-fold, 9-fold, 8-fold, 7-fold, 6-fold, 5-fold, 4-fold, 3-fold, 2-fold, or less) of the concentration on day 0 of the N-1 culture. In embodiments, the concentration of any of these molecules is measured, e.g., on any day of the N-1 culture, e.g., using standard methods.

In some embodiments, the cells are cultured in the N-1 culture for 5 to 12 days, e.g., 5, 6, 7, 8, 9, 10, 11, or 12 days.

In some embodiments, the N-1 culture is about 2 L to about 25,000 L (e.g., about 2 L to about 10 L, or about 50 L to about 100 L, or about 2000 L to about 25,000 L, or about 5,000 L to about 25,000 L, or about 10,000 L to about 25,000 L, or about 100 L to about 10,000 L, or about 100 L to about 15,000 L, or about 2000 L to about 15,000 L, or about 2000 L to about 10,000 L) in volume.

In some embodiments, the methods described herein, e.g., the fed batch process (e.g., fed batch process described herein) and/or the perfusion (e.g., ATF perfusion) process (e.g., perfusion process described herein), enable a higher seeding density (i.e., seeding the N-1 cultured cells into the N culture vessel) compared to methods that do not employ such processes for culturing the N-1 culture.

In some embodiments, the methods described herein comprise seeding the N-1 cultured cells into the N culture vessel at a density of at least 5E5 viable cells/mL, e.g., at least about 5E6 viable cells/mL, at least about 1E7 viable cells/mL, or about 5E5 viable cells/mL to about 1E7 viable cells/mL.

In some embodiments, the methods described herein comprise seeding the N-1 cultured cells into the N culture vessel at a density of about 5E5 to about 2E7 viable cells/mL, e.g., about 5E5 to about 7.5E5, about 7.5E5 to about 1E6, about 1E6 to about 2E6, about 1E6 to about 3E6, about 1E6 to about 4E6, about 1E6 to about 5E6, about 2E6 to about 4E6, about 2E6 to about 3E6, about 3E6 to about 1E7, about 3E6 to about 8E6, about 3E6 to about 6E6, about 4E6 to about 1E7, about 5E6 to about 1E7, about 6E6 to about 1E7, about 7E6 to about 1E7, about 8E6to about 1E7, about 4E6 to about 8E6, about 8E6 to about 1E7, or about 1E7 to about 2E7 viable cells/mL. These seeding densities are also referred to herein as N-1 to N (cell) seeding density. In some embodiments, the methods described herein comprise diluting the N-1 cultured cells into the N culture vessel by a dilution factor of about 1/20 to about ½, e.g., about 1/20 to about ⅓, about 1/20 to about ¼, about 1/20 to about ⅕, about 1/20 to about 1/10, about 1/20 to about 1/15, about 1/15 to about ½, about 1/15 to about ⅓, about 1/15 to about ¼, about 1/15 to about ⅕, about 1/15 to about 1/10, about 1/10 to about ½, about 1/10 to about ⅓, about 1/10 to about ¼, about 1/10 to about ⅕, about 1/10 to about ⅙, about ⅙ to about ¼, about ⅙ to about ⅓, about ⅙ to about ½, or about ⅕, about 1/7.5, about 1/10, about 1/15, or about 1/20. These dilution factors are also referred to herein as N-1 to N dilution factor.

After seeding the N culture vessel, the N culture is maintained under conditions that permit the production of AAV. In some embodiments, one or more components of AAV are added in the N culture such that the cells in the N culture are replication-competent and can produce AAV. In some embodiments, a helper virus, e.g., an adenovirus (e.g., an Ad5 helper virus) or a herpes virus, is added in the N culture.

Without wishing to be bound by theory, it is believed that the production of AAV by the helper-virus-infected (e.g., Ad5-infected) cells in the N culture leads to toxicity, decrease or elimination of cell growth, and/or cell death. As such, in some embodiments, such helper-virus-infected cells in the N culture (e.g., cells infected by Ad5 virus) exhibit a slower growth rate compared to the cells not infected by a helper virus (e.g., Ad5). In some embodiments, the cells in the N culture (e.g., the helper-virus-infected cells in the N culture) exhibit a slower growth rate compared to the cells in the N-1 culture. In some embodiments, the cells in the N culture have a lower percent cell viability compared to the cells in the N-1 culture.

In embodiments, the methods comprise culturing the cells in the N culture for less than a week, e.g., less than 7 days, e.g., less than 6 days, e.g., 5, 4, 3, 2, 1 day or less, e.g., 2-5 days, e.g., 2, 3, 4, or 5 days).

In some embodiments, the N culture is about 2000 L to about 50,000 L in volume, e.g., about 2000 L to about 40,000 L, about 2000 L to about 30,000 L, about 2000 L to about 25,000 L, about 2000 L to about 20,000 L, about 2000 L to about 15,000 L, about 2000 L to about 10,000 L, about 5000 L to about 50,000 L, about 5000 L to about 40,000 L, about 5000 L to about 30,000 L, about 5000 L to about 25,000 L, about 5000 L to about 20,000 L, about 5000 L to about 10,000 L, about 10,000 L to about 50,000 L, about 10,000 L to about 40,000 L, or about 10,000 L to about 30,000 L, in volume.

In some embodiments, the methods described herein further comprise collecting the AAV from the N culture. Standard methods of collecting or separating viral particles can be used, e.g., including but not limited to filtration or centrifugation.

In some embodiments, the methods described herein comprise determining the viable cell density of the N-1 and/or N culture periodically, e.g., at least once every 3 days, once every 2 days, once a day, twice a day, or more frequently, e.g., once per minute, two times per minute, three times per minute, four to ten times per minute, once per hour, twice per hour, three times per hour, four to ten times per hour, once per second, two times per second, three times per second, four times per second, five times per second, six times per second, seven times per second, eight times per second, nine times per second, or ten times per second.

In accordance with the methods described herein, the AAV titer, e.g., in the N culture, can be determined, e.g., on any one or more days of the N culture. An exemplary AAV titer measurement is vector genomes per cell (vg/cell). Vg/cell can be determined by standard methods in the art, e.g., including but not limited to dot blot, quantitative PCR or ddPCR, spectroscopy, or fluorimetry. See, e.g., Dorange et al. Cell Gene Therapy Insights 4.2(2018):119-129. In embodiments, the methods described herein are capable of producing (e.g., produce) an AAV titer, e.g., in the N culture, of at least about 0.5E3 vg/cell, e.g., at least about 1E4 vg/cell, about 2.5E4 vg/cell, about 5E4 vg/cell, about 1E5 vg/cell, about 2.5E vg/cell, about 5E5 vg/cell, or greater. In embodiments, the methods described herein are capable of producing (e.g., produce) an AAV titer, e.g., in the N culture, of about 1E4 vg/cell to about 6E5 vg/cell, e.g., about 1E4 to about 2E4, about 2E4 to about 4E4, about 4E4 to about 6E4, about 6E4 to about 8E4, about 8E4 to about 1E5, about 1E5 to about 2E5, about 2E5 to about 4E5, or about 4E5 to about 6E5 vg/cell. In embodiments, the methods described herein are capable of producing (e.g. produce) an AAV titer of about 1E9 vg/mL or greater, e.g., at least about 1E9 vg/mL, about 2E9 vg/mL, about 3E9 vg/mL, about 4E9 vg/mL, about 5E9 vg/mL, about 6E9 vg/mL, about 7E9 vg/mL, about 8E9 vg/mL, about 1E10 vg/mL, about 2E10 vg/mL, about 3E10 vg/mL, about 4E10 vg/mL, about 5E10 vg/mL, about 6E10 vg/mL, about 7E10 vg/mL, about 8E10 vg/mL, about 9E10 vg/mL, about 1E11 vg/mL, or greater. In embodiments, the methods described herein are capable of producing (e.g., produce) an AAV titer of about 1E9 vg/mL to about 1E11 vg/mL, e.g., about 3E9 vg/mL to about 5E10 vg/mL, about 5E9 vg/mL to about 5E10 vg/mL, about 8E9 vg/mL to about 5E10 vg/mL, about 1E10 vg/mL to about 1E11 vg/mL, about 1E10 vg/mL to about 1.5E10 vg/mL, about 1.5E10 vg/mL to about 2E10 vg/mL, about 1E10 vg/mL to about 4E10 vg/mL, about 2E10 vg/mL to about 4E10 vg/mL, or about 2E10 vg/mL to about 6E10 vg/mL.

In some embodiments, the methods described herein (e.g., using a fed batch process or a perfusion, e.g., ATF perfusion, process described herein) permit the achievement of a higher AAV titer in the N culture, e.g., compared to methods that do not employ such fed batch or perfusion process(es). In some embodiments, the methods described herein achieve an AAV titer that is at least 2-6 fold (e.g., at least 3-4 fold) that of the AAV titer of a culture process that does not comprise use of the fed batch or perfusion processes described herein. In some embodiments, the methods described herein comprise seeding the N culture at a higher seed density, e.g., which in some cases results in an AAV titer in the N culture that is at least 2-6 fold (e.g., at least 3-4 fold) that of the AAV titer of a culture process that uses a lower N-1 to N culture seed density.

Without wishing to be bound by theory, it is believed that the N-1 to N seeding density and the dilution ratio of N-1 seed culture into the N culture affects the AAV titer in the N culture.

It is believed that the seeding density may affect cell specific productivity (e.g., of AAV), where too high of a seeding density may decrease the cell specific productivity. It is believed that in some cases, the AAV titer first increases at a higher seeding density, up to a threshold level after which the AAV titer may begin to decrease, e.g., when seeding densities higher than the threshold level are used. Thus, in some embodiments, the methods described herein maximize the cell specific productivity in the N culture, e.g., by utilizing an appropriate N-1 to N cell seeding density and/or dilution factor. In some embodiments, a higher seeding density and/or dilution factor is desired. In some embodiments, a lower seeding density and/or dilution factor is desired. Depending on the desired seeding density into the N culture and the dilution ratio of the N-1 seed culture into the N culture, a fed bath process may be used for the N-1 step, or alternatively, a perfusion (e.g., ATF perfusion) process may be used for the N-1 step.

Accordingly, in some embodiments, the methods described herein comprise a fed batch process (e.g., fed batch process described herein), e.g., during the N-1 culture step. In some embodiments, the methods described herein surprisingly do not require perfusion, e.g., ATF perfusion, e.g., during the N-1 step, in order to be achieved (e.g., in order to achieve desired AAV titers, e.g., AAV titers described herein). In some embodiments, ATF perfusion is not desired, e.g., in the case where lower N-1 to N seeding densities are suitable. Thus, in some embodiments, the methods described herein do not comprise use of perfusion (e.g., ATF perfusion), e.g., during the N-1 culture step and/or the N culture step. The fed batch process advantageously is simpler and easier for technology transfer and operational strategies compared to ATF perfusion, yet may still be capable of supplying sufficient seed culture to meet the desired seeding density for AAV production (e.g., to produce AAV titers described herein).

In some embodiments, a suitable N-1 to N cell seeding density is about 1E6 to about 3E6 viable cells/mL, e.g., about 2E6 to about 3E6 viable cells/mL. In some embodiments, a suitable N-1 to N dilution factor is about ⅙ to about ⅓, e.g., about ⅕. In some embodiments, a fed batch process is used in the N-1 culture step when (i) a N-1 to N cell seeding density of about 1E6 to about 3E6 viable cells/mL is desired and/or (ii) a N-1 to N dilution factor of about ⅙ to about ⅓, e.g., about ⅕, is desired. In some embodiments, perfusion (e.g., ATF perfusion) is not used in a N-1 culture when (i) a N-1 to N cell seeding density of about 1E6 to about 3E6 viable cells/mL is desired and/or (ii) a N-1 to N dilution factor of about ⅙ to about ⅓, e.g., about ⅕, is desired. Accordingly, in some embodiments, the methods described herein comprise a fed batch process (e.g., fed batch process described herein), e.g., during the N-1 culture step, and further comprise (i) a N-1 to N seeding density of about 1E6 to about 3E6 viable cells/mL, e.g., about 2E6 to about 3E6 viable cells/mL, and/or (ii) a N-1 to N dilution factor of about ⅙ to about ⅓, e.g., about ⅕.

Without wishing to be bound by theory, it is believed that, in some embodiments, ATF perfusion is a more complex process compared to a fed batch process. In some embodiments, ATF perfusion may permit higher (e.g., at least 1.5-fold, 2-fold, 3-fold, 4-fold, 5-fold, 6-fold, 7-fold, 8-fold, 9-fold, 10-fold, or higher) N-1 to N cell seeding densities compared to a fed batch process. And, in some embodiments, higher N-1 to N seeding densities are suitable or desired. Thus, in some embodiments, the methods described herein comprise a perfusion (e.g., ATF perfusion) process, e.g., during the N-1 culture step, e.g., and achieves desired AAV titers (e.g., AAV titers described herein).

In some embodiments, a suitable N-1 to N cell seeding density is about 3E6 to about 1E7 viable cells/mL. In some embodiments, a suitable N-1 to N dilution factor is about 1/10 to about ⅕. In some embodiments, perfusion (e.g., ATF perfusion) is used in the N-1 culture step when (i) a N-1 to N cell seeding density of about 3E6 to about 1E7 viable cells/mL is desired and/or (ii) a N-1 to N dilution factor of about 1/10 to about ⅕ is desired. In some embodiments, the methods described herein comprise perfusion (e.g., ATF perfusion), e.g., during the N-1 culture step, and further comprise (i) a N-1 to N seeding density of about 3E6 to about 1E7 viable cells/mL and/or (ii) a N-1 to N dilution factor of about 1/10 to about ⅕.

### Cell Lines

Any mammalian cell line can be used in connection with the methods described herein. For example, a mammalian cell line comprising one or more AAV components (e.g., AAV components described herein), e.g., a PCL derived from any mammalian cell line, can be used in any of the methods described herein. In some embodiments, the methods described herein comprise culturing mammalian cells, e.g., human cells or non-human mammalian cells, e.g., mammalian PCLs, e.g., human PCL or non-human mammalian PCLs. Exemplary types of cells include but are not limited to: BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells +/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci., 383:44-68 (1982)); MRC 5 cells; FS4 cells; a human hepatoma line (Hep G2); and/or a PCL version of any of the cell types described herein. In some embodiments, a PCL version of a cell type described herein comprises the cell type having been engineered to possess one or more AAV components described herein.

In some embodiments, the methods described herein comprise culturing HeLa cells (e.g., HeLa PCLs), CHO cells (e.g., CHO PCLs), or HEK cells (e.g., HEK PCLs). In embodiments, the methods described herein comprise culturing HeLa cells, e.g., HeLa PCLs.

One skilled in the art will appreciate that different cell lines might have different nutrition requirements and/or might require different culture conditions for optimal growth, and will be able to modify conditions as needed.

As noted above, in some instances the cells, will be selected or engineered to include one or more AAV components (e.g., engineered into PCLs).

### Media

The cell culture(s) described herein are prepared in any medium suitable for the particular cell being cultured. In some embodiments, the medium contains e.g., inorganic salts, carbohydrates (e.g., sugars such as glucose, galactose, maltose or fructose), amino acids, vitamins (e.g., B group vitamins (e.g., B12), vitamin A vitamin E, riboflavin, thiamine and biotin), fatty acids and lipids (e.g., cholesterol and steroids), proteins and peptides (e.g., albumin, transferrin, fibronectin and fetuin), serum (e.g., compositions comprising albumins, growth factors and growth inhibitors, such as, fetal bovine serum, newborn calf serum and horse serum), trace elements (e.g., zinc, copper, selenium and tricarboxylic acid intermediates), hydrolysates (hydrolyzed proteins derived from plant or animal sources), and combinations thereof. Commercially available media such as 5×-concentrated DMEM/F12 (Invitrogen), CD OptiCHO feed (Invitrogen), CD EfficientFeed (Invitrogen), Cell Boost (HyClone), BalanCD CHO Feed (Irvine Scientific), BD Recharge (Becton Dickinson), Cellvento Feed (EMD Millipore), Ex-cell CHOZN Feed (Sigma-Aldrich), CHO Feed Bioreactor Supplement (Sigma-Aldrich), SheffCHO (Kerry), Zap-CHO (Invitria), ActiCHO (PAA/GE Healthcare), Ham's F10 (Sigma), Minimal Essential Medium ([MEM], Sigma), RPMI-1640 (Sigma), and Dulbecco's Modified Eagle's Medium ([DMEM], Sigma) are exemplary nutrient solutions. In addition, any of the media described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or 4,560,655; International Publication Nos. WO 90/03430; and WO 87/00195; the disclosures of all of which are incorporated herein by reference, can be used as culture media. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin), trace elements (defined as inorganic compounds usually present at final concentrations in the micromolar range) lipids (such as linoleic or other fatty acids) and their suitable carriers, and glucose or an equivalent energy source. In some embodiments the nutrient media is serum-free media, a protein-free media, or a chemically defined media. Any other necessary supplements can also be included at appropriate concentrations that would be known to those skilled in the art.

In accordance with any methods described herein, in embodiments, standard techniques of molecular biology, cell biology, cell culture, transgenic biology, virology, microbiology, and recombinant DNA technology, which are within the skill of the art, are utilized in connection with the methods described herein. See, e.g., such techniques as described in Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, D. N. Glover ed., Volumes I and II (1985); Oligonucleotide Synthesis, M. J. Gait ed., (1984); Mullis et al. U.S. Pat. No: 4,683,195; Nucleic Acid Hybridization, B. D. Hames & S. J. Higgins eds. (1984); Transcription And Translation, B. D. Hames & S. J. Higgins eds. (1984); Culture Of Animal Cells, R. I. Freshney, Alan R. Liss, Inc., (1987); Immobilized Cells And Enzymes, IRL Press, (1986); B. Perbal, A Practical Guide To Molecular Cloning (1984); the treatise, Methods In Enzymology, Academic Press, Inc., N.Y.; Gene Transfer Vectors For Mammalian Cells, J. H. Miller and M. P. Calos eds., Cold Spring Harbor Laboratory (1987); Methods In Enzymology, Vols. 154 and 155 (Wu et al. eds.); Immunochemical Methods In Cell And Molecular Biology, Mayer and Walker, eds., Academic Press, London (1987); Handbook Of Experimental Immunology, Volumes I-IV, D. M. Weir and C. C. Blackwell, eds., (1986); Manipulating the Mouse Embryo, Cold Spring Harbor Laboratory Press, Cold Spring Harbor, N.Y., (1986); and in Ausubel et al., Current Protocols in Molecular Biology, John Wiley and Sons, Baltimore, Maryland (1989).

The publications (including patent publications), web sites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Terms defined or used in the description and the claims shall have the meanings indicated, unless context otherwise requires. Technical and scientific terms used herein have the meaning commonly understood by one of skill in the art to which the present invention pertains, unless otherwise defined. Any conflict between an art-understood definition of a word or phrase and a definition of the word or phrase as specifically taught in this specification shall be resolved in favor of the latter.

The following examples are provided which are meant to illustrate but not limit the invention described herein.

### EXAMPLE 1

### Optimization of N-1 Seed Culture

Experiments were performed to optimize the viable cell density (VCD) that could be achieved during the N-1 seed culture. Higher N-1 VCD permitted a higher seeding density in the N culture, which enabled production of a higher titer of AAV in the N culture. In particular, two ways to supplement nutrients and remove metabolic wastes in the N-1 seed culture were tested—a fed batch process and an ATF perfusion process. Various AAV producer cell line (PCL) clones, e.g., HeLa PCL clones, were tested.

Traditional batch culture, e.g., using proprietary chemically defined media, was performed as a comparator. Using traditional batch culture methods, the viable cell density could reach about 5-6 million cells/mL, e.g., 5E6 cells/mL, before depletion of essential nutrients.

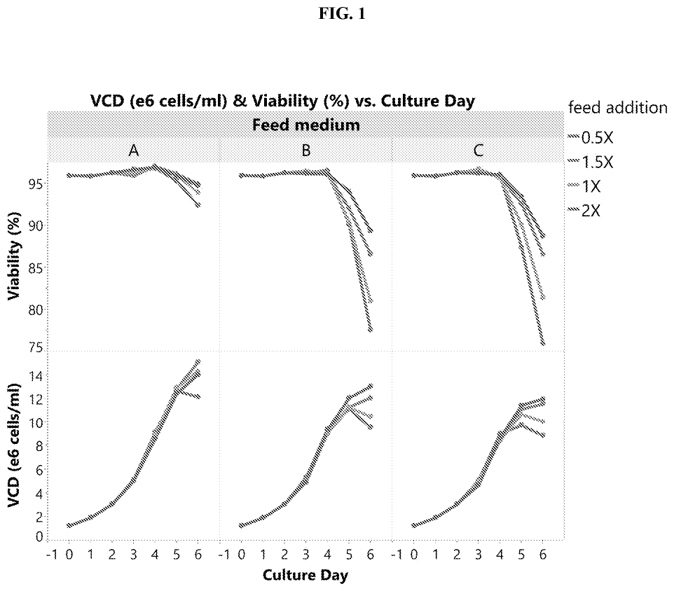

A fed batch process using an optimized feeding strategy was tested. For the fed batch process, a feed medium was added daily based on cell growth or viable cell density. In particular, feed was added daily based on integrated cell growth (ICG). ICG was calculated based on the area under the cell growth curve and was reported in units of cells*mL⁻¹*day⁻¹. Feed % was then slope*ICG. Glucose and glutamine were supplemented as needed. Using this process, a final VCD could be increased to about 1E7 cells/mL before negatively impacted by the metabolic waste accumulation. See FIG. 1. Using the fed batch process in an N-1 culture, an N culture was seeded at a higher density, e.g., about 1E6 viable cells/mL to about 5E6 viable cells/mL. The AAV titer achieved during the N culture seeded at higher cell density was compared to the AAV titer achieved during an N culture seeded at lower cell density (e.g., about 0.5E6 viable cells/mL to about 1E6 viable cells/mL). The AAV titer when using higher seed density was higher (by about 3-4 fold) than the AAV titer when using lower seed density. Using the fed batch process described herein, AAV titers of at least about 1E10 vg/mL (e.g., about 1E10 vg/mL to about 2E10 vg/mL, or about 1E10 vg/mL to about 4E10 vg/mL) were achieved.

ATF perfusion was also tested. ATF technology is believed to be scalable and to reduce shear stress to cells compared to other perfusion technologies. The ATF perfusion process is believed to permit the supplementation of nutrient while removing metabolic wastes. An exemplary ATF perfusion scheme is shown in FIG. 2. Experiments were performed to optimize several ATF perfusion parameters—perfusion medium, cell specific perfusion rate (CSPR), and ATF flow rate. Perfusion rate was increased daily based on projected cell density. The higher the CSPR, the more sufficient nutrient supply and waste removal; but too high of a CSPR could present on a burden on medium supply.

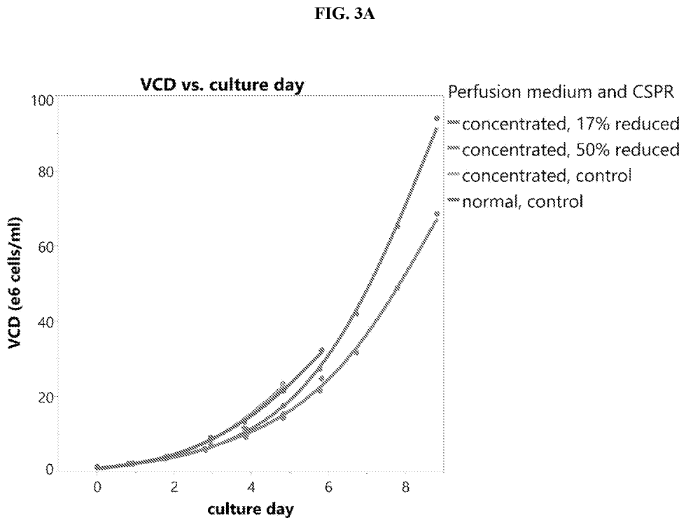

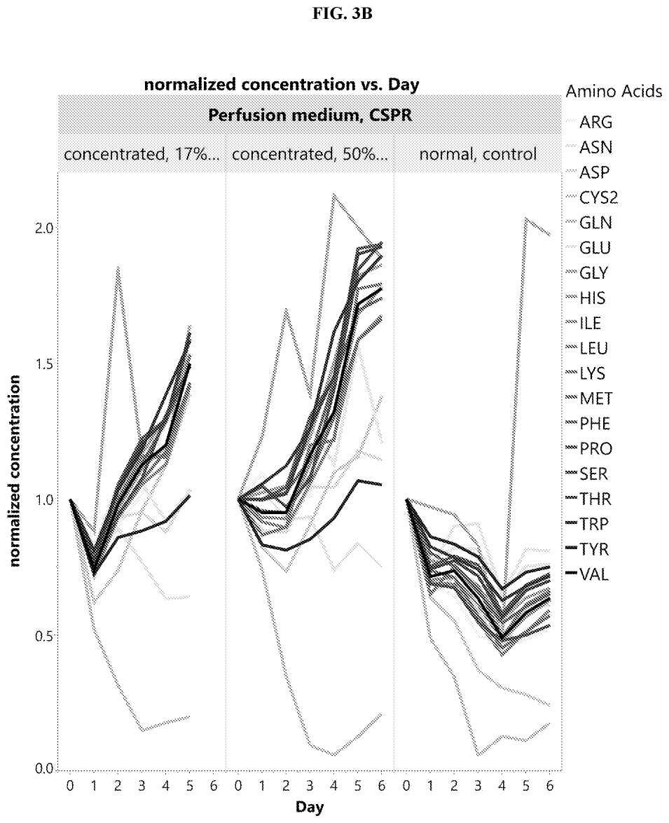

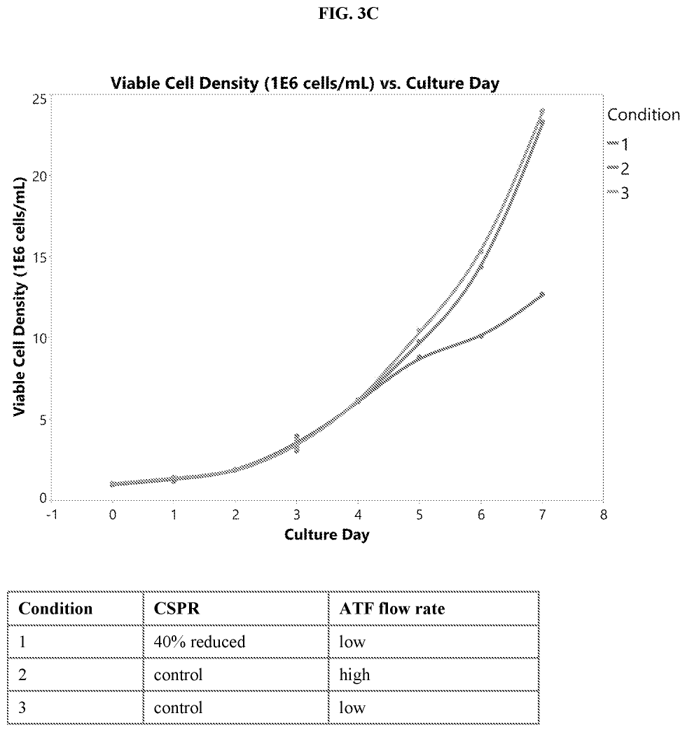

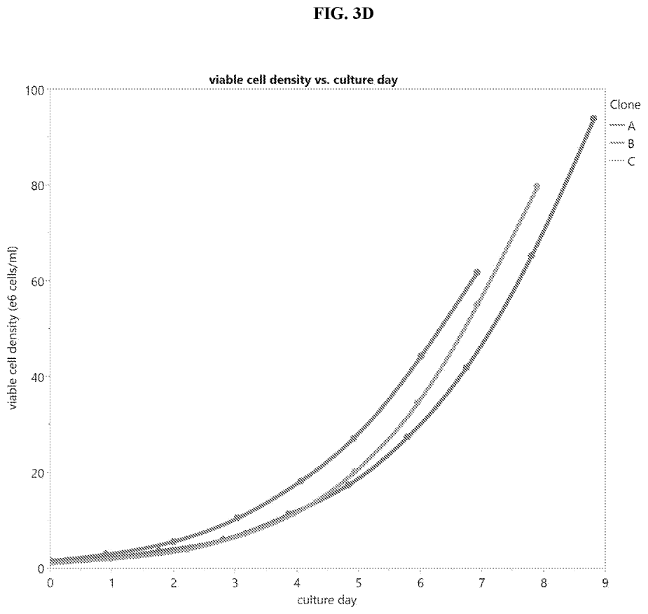

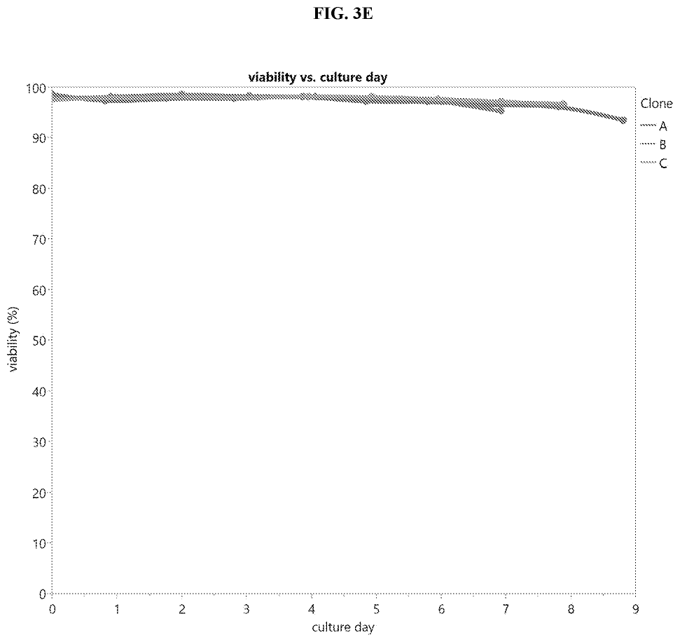

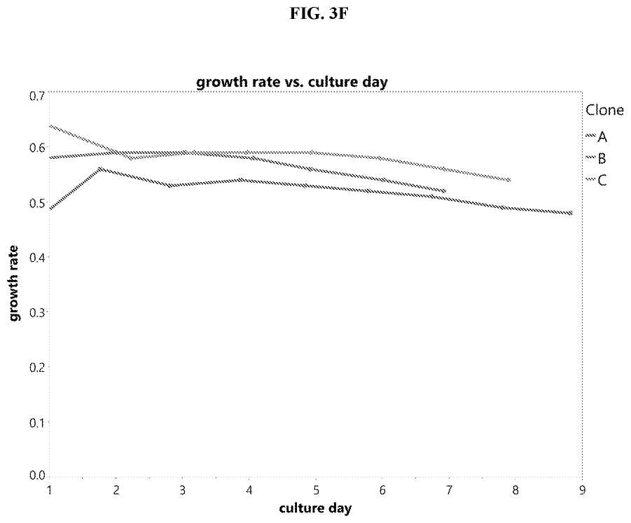

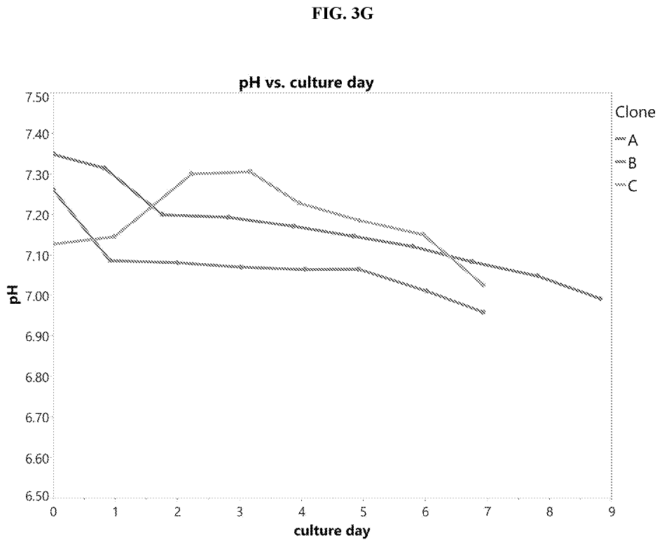

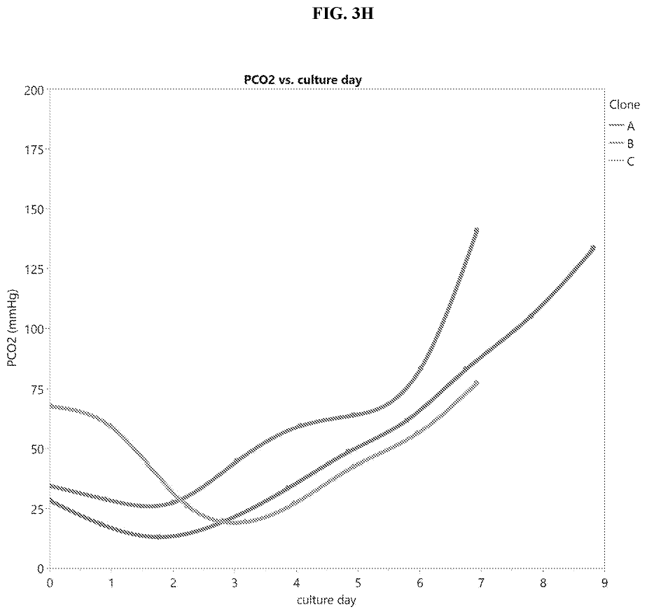

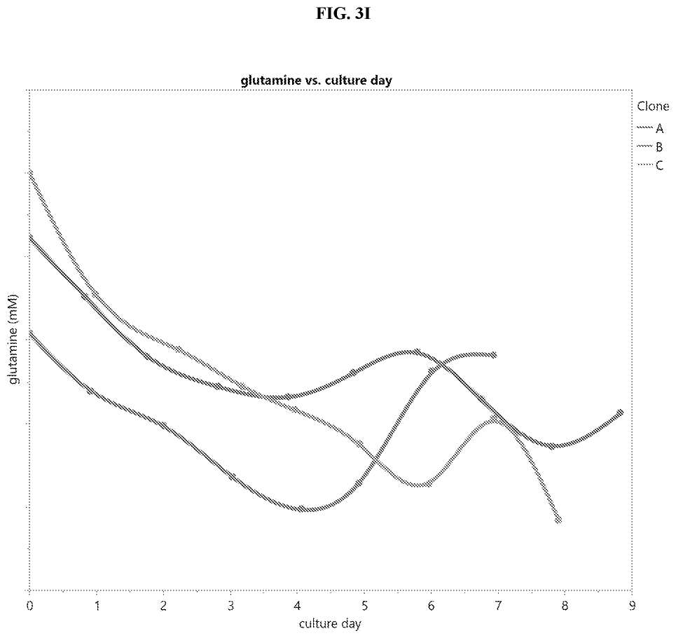

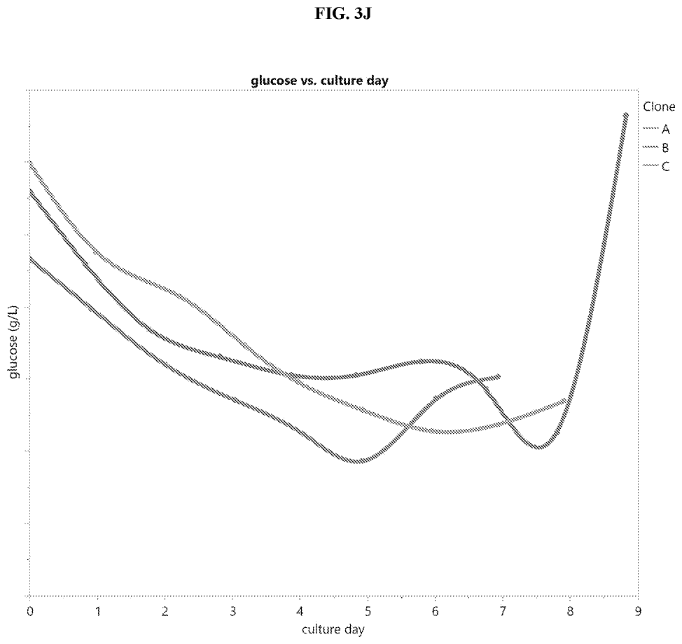

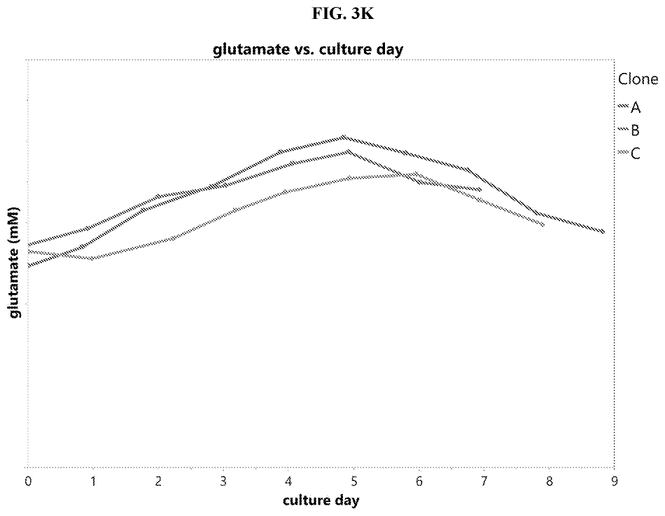

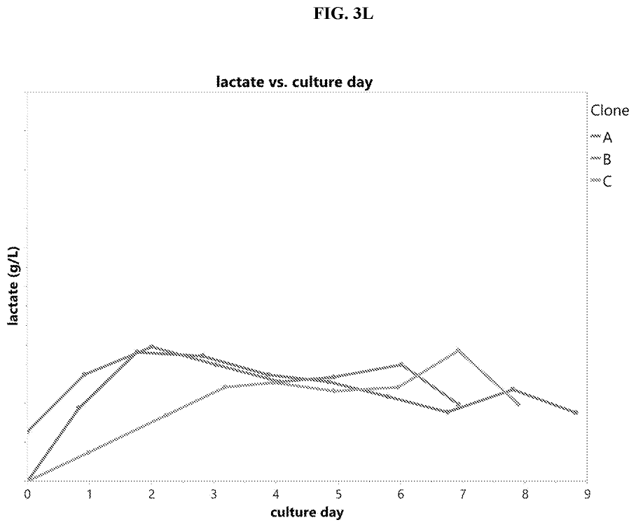

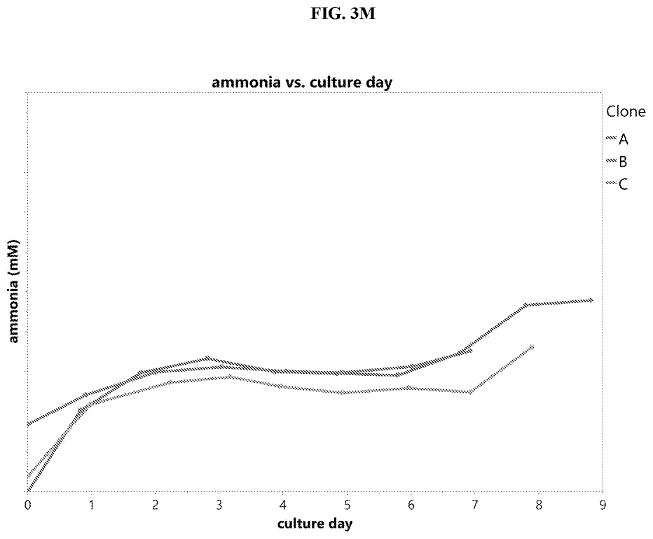

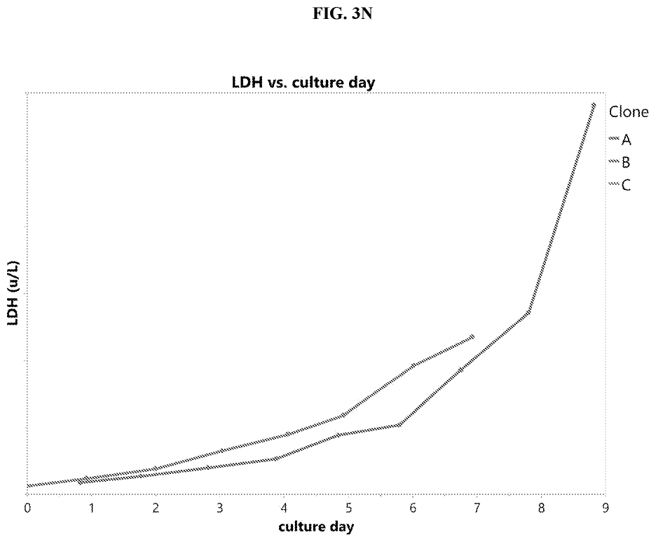

Two different perfusion media were tested, seed culture medium and a two-fold concentrated version of the seed culture medium (referred to as concentrated seed culture medium). The two media were tested at different CSPRs—CSPR reduced by 20% and CSPR reduced by 40% (where 100% CSPR refers to 0.05 nL/cell/day). At similar CSPRs, concentrated medium did not demonstrate an advantage over the original seed medium. Also, cell growth rate was inhibited when using lower CSPR and concentrated medium, though there was no indication of a severe amino acid depletion compared to using original medium at higher CSPR. See FIGS. 3A-3B.

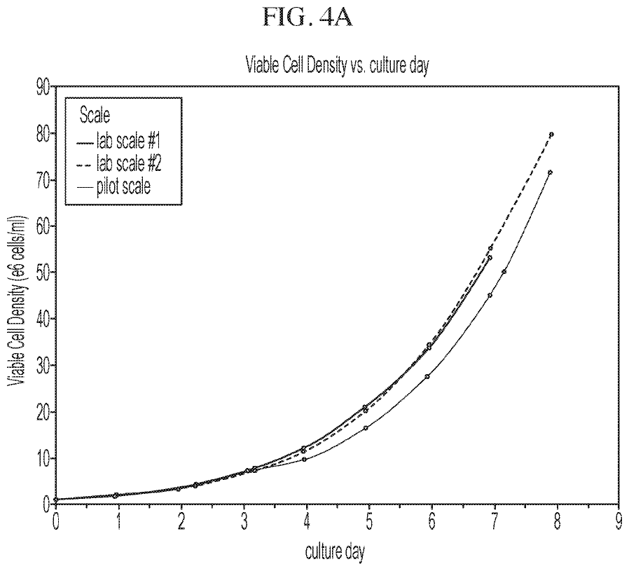

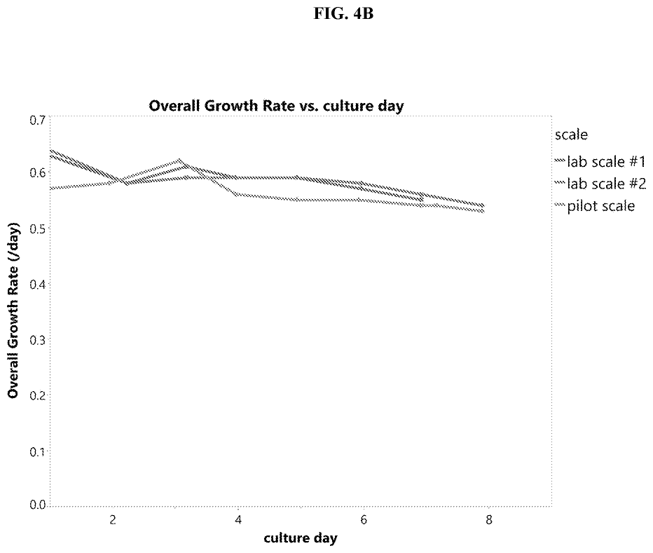

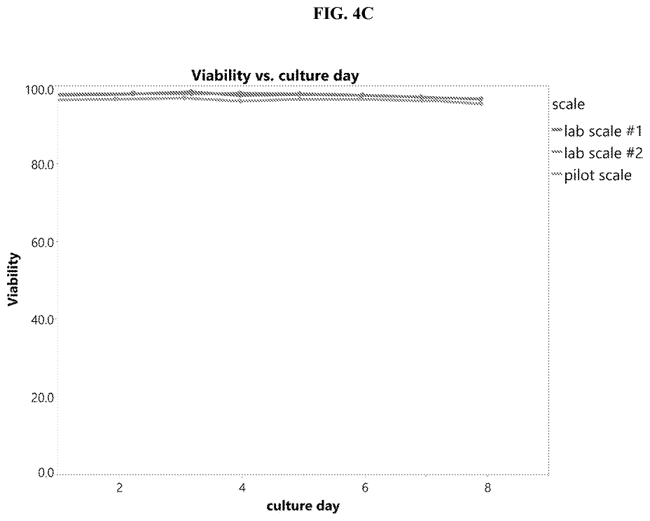

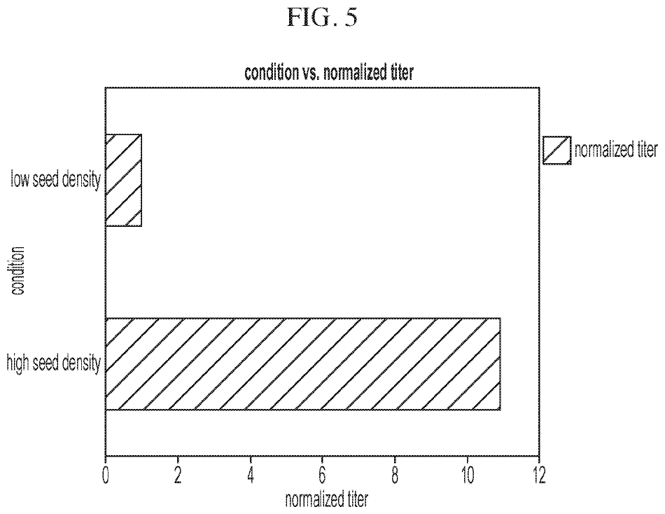

ATF flow rate was also tested. ATF flow rate is proportional to shear stress and correlates to back flush, which reduces the amount of clogging in the filter. The ATF flow rate was varied between low to high and did not significantly impact cell growth within the range of 4 mL/min/fiber to about 12 mL/min/fiber. See FIG. 3C. The ATF process was performed with multiple cell clones using an ATF flow rate of about 8 mL/min/fiber and a CSPR of 0.05 nL/cell/day; and the viability, growth rate, pH, pCO₂, nutrients (e.g., glucose, glutamine, and glutamate), waste products (e.g., lactate and ammonia), and LDH (which is correlated to cell death) were monitored on various culture days. The ATF process demonstrated robustness with multiple cell clones (FIGS. 3D-3N). The ATF process (e.g., using a flow rate per fiber of about 8 mL/min and a CSPR of about 0.04-0.05 nL/cell/day) was also robust at multiple scales, e.g., lab scale (˜1-5 L working volume) and pilot scale (˜50-150 L working volume) (FIGS. 4A-4C). A final VCD of about 1E8 cell/mL was achieved in the seed culture using the ATF process (FIG. 3A). Furthermore, by using the ATF process in the N-1 seed culture, viability and growth rate were well sustained, and nutrients were well supplied while wastes effectively removed (FIGS. 3D-3N). Additionally, a higher final VCD in the N-1 seed culture enabled an improvement in AAV genome titer in the N (AAV production) culture, relative to a lower seed density condition (an improvement of about 10-12 fold in titer). In particular, the AAV titer was about 3.4E9 vg/mL when using a seed density of about 5E5 cells/mL, and the AAV titer was about 4E10 vg/mL when using a seed density of about 2E6 cells/mL. See FIG. 5.

The embodiments of the invention described above are intended to be merely exemplary; numerous variations and modifications will be apparent to those skilled in the art. All such variations and modifications are intended to be within the scope of the present invention as defined in any appended claims.

We claim:

1. A method of producing an AAV, comprising:
(a) culturing mammalian cells (e.g., HeLa cells, CHO cells, HEK-293 cells, VERO cells, NS0 cells, PER.C6 cells, Sp2/0 cells, BHK cells, MDCK cells, MDBK cells, or COS cells) in a N-1 culture vessel, wherein the cells comprise one or more AAV components;
(b) inoculating a N culture vessel with the cells obtained from step (a) at a predetermined dilution factor; and
(c) culturing the cells in the N culture vessel under conditions that allow production of the AAV,
wherein step (a) further comprises removing waste products and/or supplementing with fresh nutrients, e.g., using perfusion (e.g., alternating tangential flow (ATF) perfusion) or using a fed batch process,
thereby producing the AAV.

2. The method of claim 1, wherein the step (a) comprises using a fed batch process.

3. The method of claim 2, wherein
(i) the predetermined dilution factor in the step (b) is about ⅙ to about ⅓, e.g., about ⅕, of the cell density of the cells obtained from step (a); and/or
(ii) the N culture vessel seeding density of step (b) is about 1E6 to about 3E6 viable cells/mL, e.g., about 2E6 to about 3E6 viable cells/mL.

4. The method of claim 1, wherein the step (a) comprises using ATF perfusion.

5. The method of claim 4, wherein
(i) the predetermined dilution factor in the step (b) is about 1/10 to about ⅕ of the cell density of the cells obtained from step (a); and/or
(ii) the N culture vessel seeding density of step (b) is about 3E6 to about 1E7 viable cell s/mL.

6. The method of any of the previous claims, wherein the predetermined dilution factor in the step (b) is about 1/20 to about ½ of the cell density of the cells obtained from step (a).

7. The method of claim 6, wherein the predetermined dilution factor in the step (b) is about ⅙ to about ⅓, e.g., about ⅕ of the cell density of the cells obtained from step (a),
optionally wherein the step (a) comprises using a fed batch process, e.g., a fed batch process described herein; and/or
optionally wherein the step (a) does not comprise using perfusion, e.g., ATF perfusion.

8. The method of claim 6, wherein the predetermined dilution factor in the step (b) is about 1/10 to about ⅕ of the cell density of the cells obtained from step (a),
optionally wherein the step (a) comprises using perfusion, e.g., ATF perfusion, e.g., ATF perfusion described herein.

9. The method of any of the previous claims, wherein the step (c) is performed for less than 6 days (e.g., 5, 4, 3, 2, 1 day or less, e.g., 2-5 days, e.g., 2, 3, 4, or 5 days).

10. The method of any of the previous claims, wherein the step (a) comprises culturing the cells to achieve an N-1 cell density of at least 1E7 viable cells/mL, e.g., about 1E7 to about 2E8 viable cells/mL, e.g., about 2E7 to about 1E8.

11. The method of any of the previous claims, wherein the N culture vessel seeding density of step (b) is about 5E5 to about 2E7 viable cells/mL.

12. The method of claim 11, wherein the N culture vessel seeding density of step (b) is about 1E6 to about 3E6 viable cells/mL,
optionally wherein the step (a) comprises using a fed batch process, and
optionally wherein the step (a) does not comprise using perfusion.

13. The method of claim 11, wherein the N culture vessel seeding density of step (b) is about 3E6 to about 1E7 viable cells/mL,
optionally wherein the step (b) comprises using perfusion, e.g., ATF perfusion.

14. The method of any of the previous claims, wherein the N-1 culture is about 2 L to about 25,000 L (e.g., about 2 L to about 10 L, or about 50 L to about 100 L, or about 2000 L to about 25,000 L) in volume.

15. The method of any of the previous claims, wherein the N culture is about 2000 L to about 50,000 L in volume.

16. The method of any of the previous claims, wherein the cells comprise HeLa cells, e.g., HeLa PCLs.

17. The method of any of the previous claims, wherein the step (a) and step (c) comprise culturing the cells in suspension.

18. The method of any of the previous claims, wherein the ATF perfusion is performed at a flow rate of about 3 mL/min/fiber to about 15 mL/min/fiber.

19. The method of any of the previous claims, wherein the cell specific perfusion rate (CSPR) in the step (a) is at least about 0.02 nL/cell/day, e.g., about 0.02 nL/cell/day to about 0.1 nL/cell/day, e.g., about 0.03 nL/cell/day to about 0.06 nL/cell/day, e.g., about 0.03 nL/cell/day, about 0.04 nL/cell/day, about 0.05 nL/cell/day, or about 0.06 nL/cell/day.

20. The method of any of the previous claims, wherein the step (a) is performed for 5 to 12 days.

21. The method of any of the previous claims, further comprising a step (d) collecting the AAV produced by the cells in the N culture vessel.

22. The method of any of the previous claims, wherein the AAV component comprises one or more (any combination) of the following:
(a) a nucleic acid sequence comprising a transgene;
(b) a nucleic acid sequence comprising an inverted terminal repeat (ITR), e.g., one or two ITRs;
(c) a nucleic acid sequence encoding one or more AAV replication and/or packaging proteins (e.g., encoded by the AAV rep gene);
(d) a nucleic acid sequence encoding one or more AAV structural capsid proteins (e.g., encoded by the AAV cap gene, e.g., VP1, VP2, or VP3 protein);
(e) one or more AAV replication and/or packaging proteins;
(f) one or more AAV structural capsid proteins; and/or
(g) one or more helper virus components, e.g., Ad5 helper function components (e.g., Ad5 helper function components described herein, e.g., Ad5 helper virus, or E1a, E1b, E2a, E4Orf6, and/or VA RNA),
optionally wherein any combination of (a)-(d) or (g) are disposed on the same nucleic acid molecule or on separate nucleic acid molecules (e.g., disposed on one, two, three, or four separate nucleic acid molecules).

23. The method of any of the previous claims, wherein the step (c) further comprises providing a helper virus, e.g., an adenovirus (e.g., Ad5 helper virus) or a herpes virus, to the N culture vessel.

24. The method of any of the previous claims, wherein the step (c) further comprises providing one or more additional AAV components (e.g., AAV components described herein, e.g., AAV components that are not already in the N-1 culture) to the N culture vessel.

25. The method of any of claims 22-24, wherein the transgene encodes a therapeutic polypeptide.

26. The method of any of the previous claims, wherein the fed batch process comprises supplementing the N-1 culture periodically with a supplement (e.g., fresh media, amino acids, and/or glucose).

27. The method of claim 26, wherein the fed batch process comprises supplementing the N-1 culture once a day, every 2 days, or twice a day.

28. The method of claim 27, wherein the fed batch process comprises supplementing the N-1 culture once a day.

29. The method of any of claims 26-28, wherein the amount (e.g., volume, concentration, and/or feed %) of the supplement is determined based on the integrated cell growth (ICG) of the N-1 culture.

30. The method of any of claims 26-29, wherein the amount (e.g., volume, concentration, and/or feed %) of the supplement is determined using a feed addition slope of about 0.0002 mL*day/cells to about 0.02 mL*day/cells, e.g., about 0.0005 mL*day/cells to about 0.005 mL*day/cells.
